(19) United States
(12) Patent Application Publication
(10) Pub. No.: US 2024/0343680 A1
(43) Pub. Date: Oct. 17, 2024

(54) PROCESS FOR PREPARING ACYL DERIVATIVES

(71) Applicants: Centre National de la Recherche Scientifique, Paris (FR); Universite Claude Bernard Lyon 1, Villeurbanne (FR); Institut National des Sciences Appliquees de Lyon, Villeurbanne (FR)

(72) Inventors: Estelle Metay, Communay (FR); Marc Lemaire, Villeurbanne (FR); Marie-Christine Duclos, Villeurbanne (FR); Marc Daumas, Eyragues (FR); Olivier Pardigon, Mison (FR)

(73) Assignees: Centre National de la Recherche Scientifique, Paris (FR); Universite Claude Bernard Lyon 1, Villeurbanne (FR); Institut National des Sciences Appliquees de Lyon, Villeurbanne (FR)

(21) Appl. No.: 18/702,628

(22) PCT Filed: Oct. 27, 2022
(86) PCT No.: PCT/EP2022/080036

§ 371 Date: Apr. 18, 2024

(30) Foreign Application Priority Data
Oct. 28, 2021 (EP) 21306519.6

(51) Int. Cl. C07C 233/25; C07C 50/02

(52) U.S. Cl. CPC C07C 233/25; C07C 50/02

(57) ABSTRACT

The present invention relates to a process for preparing aromatic acyl derivatives of formula (I) using methanesulfonic acid. The invention further relates to methods for preparing biological active ingredients. such as paracetamol.

### FIELD OF THE INVENTION

The present invention relates to the organic chemistry field. More particularly, it relates to improved methods for preparing aromatic acyl derivatives, which can be useful as intermediates for the synthesis of biological active ingredients, such as paracetamol and ibuprofen.

### BACKGROUND OF THE INVENTION

To access to biological active ingredients, one of the most important reactions from an industrial synthetic point of view is the acylation of aromatic substrates. This electrophilic aromatic substitution is generally carried out in the presence of large quantities of inorganic salts inducing a corresponding quantity of toxic and corrosive wastes. In most cases, toxic solvents (chlorinated, aromatic, or both) have to be used.

For instance, acylation reactions generally involve the use of Lewis acids such as AlCl₃, FeCl₃, SnCl₄ or rare-earth triflates. However, these Lewis acids must be used in large quantities, and are expensive, toxics, and not recyclable. Acylation reactions may also be performed using Brönsted acids, such as fluorhydric acid, trifluoroacetic acid, and triflic acid. However, similarly to Lewis acids, such fluorinated reagents are expensive, toxics, and dangerous to manipulate.

In order to have access to several important intermediates for the industrial synthesis of drugs, there remains a need to improve the acylation reaction with good yields, high selectivity and low ecological impact suitable for the industrial scale that includes economic, ecological and safety objectives.

### SUMMARY OF THE INVENTION

In this context, the inventors have studied and developed new methods for preparing aromatic acyl derivatives. Unexpectedly, the inventors have shown that the use of methanesulfonic acid in acylation reactions allows to obtain aromatic acyl derivatives with good yields and high selectivity. Such a use of methanesulfonic acid is compatible with the industrial approach since it is readily available, easier and less dangerous to manipulate compared to the fluorinated acid and Lewis acid, cost-effective, and avoids the use of expensive starting materials and large amounts of reagents having a high ecological impact. These acylation methods may be used in the synthesis of biological active ingredients. For instance, the inventors have implemented an acylation reaction using methanesulfonic acid for preparing paracetamol. The inventors have further improved a process for preparing paracetamol starting from hydroquinone.

Accordingly, the present invention relates to a process for preparing a compound of formula (I):

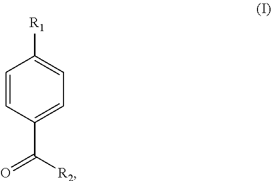

in which:
R₁ is a radical selected in the group consisting of:
   a hydroxy group,
   a (C₁-C₁₈)alkyl group,
   a (C₁-C₆)alkoxy group, and
   a halogen group;
R₂ is a radical selected in the group consisting of:
   a (C₁-C₁₈)alkyl group,
   a phenyl optionally substituted by at least one hydroxy group, and
   a (C₁-C₆)alkoxy group;
      comprising the following steps of:
a) reacting a compound of formula (II):

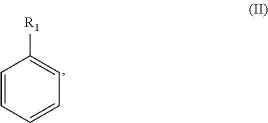

R₁ being such as above defined,
   with methane sulfonic acid and a compound of formula (III):

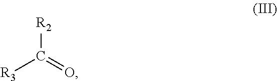

in which:
R₂ is such as above defined, and
R₃ is a radical selected in the group consisting of a hydroxy, a —O—CO—CH₃ group, a (C₁-C₆)alkoxy group, and a chlorine; and
   b) recovering said compound of formula (I).

Preferably, R₂ is a radical selected in the group consisting of:
a (C₁-C₁₈)alkyl group,
a phenyl, and
a (C₁-C₆)alkoxy group.

In a particular embodiment, the reaction at step a) is carried out at a temperature from 30° C. to 130° C., preferably from 40° C. to 60° C., more preferably about 50° C. In a further particular embodiment 1 to 5 equivalents, preferably 1 to 3 equivalents, more preferably 1 to 2.5 equivalents, even more preferably 1, 1.5 or 2.5 equivalents of the compound of formula (III) relative to the compound of formula (II) is used at step a).

In a preferred embodiment, the compound of formula (I) is such that R₁ is a hydroxy group and R₂ is a methyl group, the compound of formula (II) is such that R₁ is a hydroxy group, and the compound of formula (III) is such that R₂ is a methyl group and R₃ is a hydroxy group.

A preferred process of the invention thus comprises the following steps of:
a) reacting phenol with methane sulfonic acid and acetic acid at a temperature about 50° C. in which 1 to 5 equivalents, preferably 1 to 3 equivalents, more preferably 1 to 2.5 equivalents, even more preferably 1, 1.5 or 2.5 equivalents of acetic acid relative to phenol is used; and
b) recovering 4-hydroxyacetophenone.

Another object of the invention is a process for preparing paracetamol comprising the following steps of:
a)-b) implementing the process as above defined;
c) reacting 4-hydroxyacetophenone with formic acid and hydrogen peroxide;
d) reacting the mixture obtained after step c) with ammonium acetate and acetic acid; and
e) recovering paracetamol.

Preferably, the process for preparing paracetamol comprises the following steps of:
a) reacting phenol with methane sulfonic acid and acetic acid at a temperature about 50° C., in which 2.5 equivalents of acetic acid relative to phenol is used;
b) recovering 4-hydroxyacetophenone;
c) reacting 4-hydroxyacetophenone with formic acid and hydrogen peroxide;
d) reacting the mixture obtained after step c) with ammonium acetate and acetic acid; and
e) recovering paracetamol.

In a particular embodiment, such a process for preparing paracetamol further comprises a step of purifying the mixture obtained after step c) to recover hydroquinone.

A further object of the invention is a process for preparing paracetamol comprising the following steps of:
reacting hydroquinone with ammonium acetate or acetamide and water at a temperature ranging from 240 to 300° C. for a period from 1 min to 12 hours in which 1 to 50 equivalents of ammonium acetate or acetamide and water relative to hydroquinone are used, wherein the reaction is carried out in the absence of acetic acid; and
recovering paracetamol.

In a particular embodiment, hydroquinone and ammonium acetate are reacted at a temperature about 260° C. for about 1 hour in which 10 equivalents of ammonium acetate relative to hydroquinone are used.

In a particular embodiment, hydroquinone, acetamide, and water are reacted at a temperature about 260° C. for about 1 hour in which 10 equivalents of acetamide and 10 equivalents of water, relative to hydroquinone are used.

In a further preferred embodiment, the process for preparing a compound of formula (I) is such that the compound of formula (I) is such that R₁ is a (C₁-C₆)alkyl group, preferably an isobutyl group and R₂ is a methyl group, the compound of formula (II) is such that R₁ is a (C₁-C₆)alkyl group, preferably an isobutyl group, and the compound of formula (III) is such that R₂ is a methyl group and R₃ is a —O—CO—CH₃ group.

A preferred process of the invention thus comprises the following steps of:
a) reacting 4-isobutylbenzene with methane sulfonic acid and acetic anhydride at a temperature about 50° C. in which 2 equivalents of acetic anhydride relative to 4-iso-butylphenyl is used; and
b) recovering preferably 1-(4-isobutylphenyl)ethanone.

Another object of the invention is a process for preparing ibuprofen comprising the following steps:
a) preparing 1-(4-isobutylphenyl)ethanone as above defined; and
b) obtaining ibuprofen from the intermediate 1-(4-isobutylphenyl)ethanone recovered at step a).

In another preferred embodiment, the process for preparing a compound of formula (I) is such that:
the compound of formula (I) is such that R₁ is a radical selected in the group consisting of a hydroxy group, a methoxy group, and a chlorine, and R₂ is a radical selected in the group consisting of an octyl group, and a phenyl optionally substituted by at least one hydroxy group, preferably three hydroxy groups,
the compound of formula (II) is such that R₁ is a radical selected in the group consisting of a hydroxy group, a methoxy group, and a chlorine, and
the compound of formula (III) is such that R₂ is a radical selected in the group consisting of an octyl group, and a phenyl optionally substituted by at least one hydroxy group, preferably three hydroxy groups, and R₃ is a hydroxy or a chlorine.

### DETAILED DESCRIPTION

### Definitions

According to the present invention, the terms below have the following meanings:

The terms mentioned herein with prefixes such as for example C₁-C₁₈, can also be used with lower numbers of carbon atoms such as C₁-C₁₂, C₁-C₆, or C₁-C₂. If, for example, the term C₁-C₁₂ is used, it means that the corresponding hydrocarbon chain may comprise from 1 to 12 carbon atoms, especially 1, 2, 3, 4, 5, 6, 7, 8, 9, 10, 11, or 12 carbon atoms. If, for example, the term C₁-C₆ is used, it means that the corresponding hydrocarbon chain may comprise from 1 to 6 carbon atoms, especially 1, 2, 3, 4, 5, or 6 carbon atoms. If, for example, the term C₁-C₃ is used, it means that the corresponding hydrocarbon chain may comprise from 1 to 3 carbon atoms, especially 1, 2, or 3 carbon atoms.

The term “alkyl” refers to a saturated, linear or branched aliphatic group. The term “(C₁-C₁₂)alkyl” more specifically means methyl, ethyl, propyl, isopropyl, butyl, isobutyl, pentyl, isopentyl, hexyl, heptyl, hexyl, nonyl, decyl, undecyl, or dodecyl. The term “(C₁-C₆)alkyl” more specifically means methyl, ethyl, propyl, isopropyl, butyl, isobutyl, pentyl, isopentyl, or hexyl.

The term “alkoxy” or “alkyloxy” corresponds to the alkyl group as above defined bonded to the molecule by an —O— (ether) bond. (C₁-C₆)alkoxy includes methoxy or methyloxy, ethoxy or ethyloxy, propoxy or propyloxy, isopropoxy or isopropyloxy, butoxy or butyloxy, isobutoxy or isobutyloxy, pentoxy or pentyloxy, isopentoxy or isopentyloxy, and hexoxy hexyloxy.

The term “halogen” corresponds to a fluorine, chlorine, bromine, or iodine atom, preferably a chlorine.

The expressions “a radical substituted by a” and “a radical substituted by at least” means that the radical is substituted by one or several groups of the list. For instance, the expression “a phenyl substituted by at least one hydroxy group” may include a phenyl substituted by one, two, three, four, and five hydroxy groups, preferably three hydroxy groups.

As used herein, the terms “active principle”, “active ingredient”, “active pharmaceutical ingredient”, “biological active ingredient”, and “drug” are equivalent and refers to a component of a pharmaceutical composition having a therapeutic effect. As an example, paracetamol and ibuprofen may be cited.

As used herein, the term “about” will be understood by a person of ordinary skill in the art and will vary to some extent on the context in which it is used. If there are uses of the term which are not clear to persons of ordinary skill in the art given the context in which it is used, “about” will mean up to plus or minus 20%, preferably 10% of the particular term.

### Acylation

The present invention provides a process for preparing aromatic acyl derivatives comprising reacting an aromatic derivative with methane sulfonic.

More particularly, the present invention provides a process for preparing a compound of formula (I):

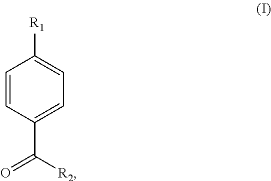

in which:
R₁ is a radical selected in the group consisting of:
   a hydroxy group,
   a (C₁-C₁₈)alkyl group, preferably a (C₁-C₁₂)alkyl group, more preferably a (C₁-C₁₂)alkyl group,
   a (C₁-C₆)alkoxy group, and
   a halogen group;
R₂ is a radical selected in the group consisting of:
   a (C₁-C₁₈)alkyl group, preferably a (C₁-C₁₂)alkyl group,
   a phenyl optionally substituted by at least one hydroxy group, and
   a (C₁-C₆)alkoxy group;
      comprising the following steps of:
a) reacting a compound of formula (II):

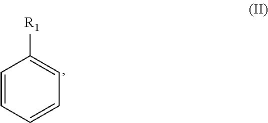

R₁ being such as above defined,
   with methane sulfonic acid and a compound of formula (III):

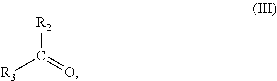

in which:
R₂ is such as above defined, and
R₃ is a radical selected in the group consisting of a hydroxy, a —O—CO—CH₃ group, a (C₁-C₆)alkoxy group, and a chlorine; and
   b) recovering said compound of formula (I).

In a preferred embodiment, the present invention provides a process for preparing a compound of formula (I):

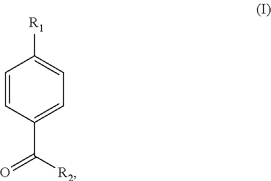

in which:
R₁ is a radical selected in the group consisting of:
   a hydroxy group,
   a (C₁-C₁₈)alkyl group, preferably a (C₁-C₁₂)alkyl group, more preferably a (C₁-C₁₂)alkyl group,
   a (C₁-C₆)alkoxy group, and
   a halogen group;
R₂ is a radical selected in the group consisting of:
   a (C₁-C₁₈)alkyl group, preferably a (C₁-C₁₂)alkyl group,
   a phenyl, and
   a (C₁-C₆)alkoxy group;
      comprising the following steps of:
a) reacting a compound of formula (II):

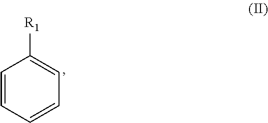

R₁ being such as above defined,
   with methane sulfonic acid and a compound of formula (III):

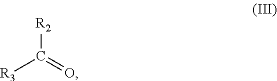

in which:
R₂ is such as above defined, and
R₃ is a radical selected in the group consisting of a hydroxy, a —O—CO—CH₃ group, a (C₁-C₆)alkoxy group, and a chlorine; and
   b) recovering said compound of formula (I).

In a preferred embodiment, R₂ is a radical selected in the group consisting of:
a (C₁-C₁₈)alkyl group,
a (C₁-C₆)alkoxy group.

In a particular embodiment, the reaction at step a) is carried out at a temperature from 30° C. to 130° C., preferably from 40° C. to 60° C., more preferably about 50° C.

In a further particular embodiment, the reaction at step a) is carried out for which 1 to 5 equivalents, preferably 1 to 3 equivalents, more preferably 1 to 2.5 equivalents, even more preferably 1, 1.5 or 2.5 equivalents of the compound of formula (III) relative to the compound of formula (II) is used.

In a further preferred embodiment, the compound of formula (I) is such that R₁ is a hydroxy group and R₂ is a methyl group, the compound of formula (II) is such that R₁ is a hydroxy group, and the compound of formula (III) is such that R₂ is a methyl group and R₃ is a hydroxy group.

According to this preferred embodiment, the process comprises the following steps:
a) reacting phenol with methane sulfonic acid and acetic acid at a temperature about 50° C. in which 1 to 5 equivalents, preferably 1 to 3 equivalents, more preferably 1 to 2.5 equivalents, even more preferably 1, 1.5 or 2.5 equivalents of acetic acid relative to phenol is used; and
b) recovering 4-hydroxyacetophenone.

In a further preferred embodiment, the compound of formula (I) is such that R₁ is a (C₁-C₆)alkyl group, preferably an isobutyl group, and R₂ is a methyl group, the compound of formula (II) is such that R₁ is a (C₁-C₆)alkyl group, preferably an isobutyl group, and the compound of formula (III) is such that R₂ is a methyl group and R₃ is a —O—CO—CH₃ group.

A preferred process thus comprises the following steps:
a) reacting 4-isobutylbenzene with methane sulfonic acid and acetic anhydride at a temperature about 50° C. in which 1 to 5 equivalents, preferably 1 to 3 equivalents, more preferably 2 equivalents of acetic anhydride relative to 4-iso-butylphenyl is used; and
b) recovering 1-(4-isobutylphenyl)ethanone.

In a further preferred embodiment, the compound of formula (I) is such that R₁ is a hydroxy group and R₂ is an octyl group, the compound of formula (II) is such that R₁ is a hydroxy group, and the compound of formula (III) is such that R₂ is an octyl group and R₃ is a hydroxy group. A preferred process thus comprises the following steps:
a) reacting phenol with methane sulfonic acid and nonanoic acid at a temperature about 50° C. in which 1 to 5 equivalents, preferably 1 to 3 equivalents, more preferably 1 to 2.5 equivalents, even more preferably 1, 1.5 or 2.5 equivalents of nonanoic acid relative to phenol is used; and
b) recovering 1-(4-hydroxy-phenyl)nonan-1-one.

In a further preferred embodiment, the compound of formula (I) is such that R₁ is a hydroxy group and R₂ is a phenyl group, the compound of formula (II) is such that R₁ is a hydroxy group, and the compound of formula (III) is such that R₂ is a phenyl group and R₃ is a hydroxy group. A preferred process thus comprises the following steps:
a) reacting phenol with methane sulfonic acid and benzoic acid at a temperature about 60° C. in which 1 to 5 equivalents, preferably 1 to 3 equivalents, more preferably 1 to 2.5 equivalents, even more preferably 1, 1.5 or 2.5 equivalents of benzoic acid relative to phenol is used; and
b) recovering 4-hydroxybenzophenone.

In a further preferred embodiment, the compound of formula (I) is such that R₁ is a methoxy group and R₂ is a phenyl group, the compound of formula (II) is such that R₁ is a methoxy group, and the compound of formula (III) is such that R₂ is a phenyl group and R₃ is a hydroxy group.

A preferred process thus comprises the following steps:
a) reacting anisole with methane sulfonic acid and benzoic acid at a temperature about 60° C. in which 1 to 5 equivalents, preferably 1 to 3 equivalents, more preferably 1 to 2.5 equivalents, even more preferably 1, 1.5 or 2.5 equivalents of benzoic acid relative to anisole is used; and
b) recovering 4-methoxy benzophenone.

In a further preferred embodiment, the compound of formula (I) is such that R₁ is a hydroxy group and R₂ is a phenyl group substituted by three hydroxy groups (i.e. gallic acid), the compound of formula (II) is such that R₁ is a hydroxy group, and the compound of formula (III) is such that R₂ is a phenyl group substituted by three hydroxy groups and R₃ is a hydroxy group. A preferred process thus comprises the following steps:
a) reacting phenol with methane sulfonic acid and 3,4,5-trihydroxybenzoic acid at a temperature about 120° C. in which 1 to 5 equivalents, preferably 1 to 3 equivalents, more preferably 1 to 2.5 equivalents, even more preferably 1, 1.5 or 2.5 equivalents of 3,4,5-trihydroxybenzoic acid relative to phenol is used; and
b) recovering (4-hydroxyphenyl)-(3,4,5-trihydroxyphenyl) methanone.

In a further preferred embodiment, the compound of formula (I) is such that R₁ is a chlorine and R₂ is a phenyl group, the compound of formula (II) is such that R₁ is a chlorine, and the compound of formula (III) is such that R₂ is a phenyl group and R₃ is a chlorine. A preferred process thus comprises the following steps:
a) reacting chlorobenzene with methane sulfonic acid and benzoyl chloride at a temperature about 120° C. in which 1 to 5 equivalents, preferably 1 to 3 equivalents, more preferably 1 to 2.5 equivalents, even more preferably 1, 1.5 or 2.5 equivalents of benzoyl chloride relative to chlorobenzene is used; and
b) recovering 4-chlorobenzophenone.

Particularly, the aromatic acyl derivatives are prepared in a single chemical step, without considering the recovering step. The processes of the invention are thus more suitable for an industrial scale compared to processes and methods using Fries rearrangement for preparing 2-hydroxyacteophenone which imposes a previous step for preparing of acetyl benzene, such as those disclosed by Hocking (J. Chem. Tech. Biotechnol., 1980, 30, 626-641).

As used herein, the term “comprise(s)” or “comprising” (and other comparable terms, e.g., “containing.” and “including”) is “open-ended” and can be generally interpreted such that all of the specifically mentioned features and any optional, additional and unspecified features are included. According to specific embodiments, it can also be interpreted as the phrase “consisting essentially of” where the specified features and any optional, additional and unspecified features that do not materially affect the basic and novel characteristic(s) of the claimed invention are included or the phrase “consisting of”' where only the specified features are included, unless otherwise stated.

Thus, an object of the invention also relates to a process for preparing a compound of formula (I):

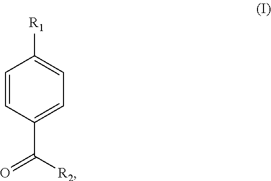

in which:
R₁ is a radical selected in the group consisting of:
   a hydroxy group,
   a (C₁-C₁₈)alkyl group, preferably a (C₁-C₁₂)alkyl group, more preferably a (C₁-C₁₂)alkyl group,
   a (C₁-C₆)alkoxy group, and
   a halogen group;
R₂ is a radical selected in the group consisting of:
   a (C₁-C₁₈)alkyl group, preferably a (C₁-C₁₂)alkyl group,
   a phenyl optionally substituted by at least one hydroxy group, preferably a phenyl, and
   a (C₁-C₆)alkoxy group;
      consisting of the following steps of:
a) reacting a compound of formula (II):

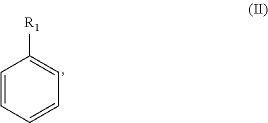

R₁ being such as above defined,
   with methane sulfonic acid and a compound of formula (III):

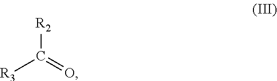

in which:
R₂ is such as above defined, and
R₃ is a radical selected in the group consisting of a hydroxy, a —O—CO—CH₃ group, a (C₁-C₆)alkoxy group, and a chlorine; and
   b) recovering said compound of formula (I), including all the particular and preferred embodiments as disclosed herein.

As above defined and illustrated by the following examples, the inventors have developed a novel approach for preparing aromatic acyl derivatives using methane sulfonic acid. Such aromatic acyl derivatives may be used as intermediates for the synthesis of a large number of arylketone derivatives having a potential biological or therapeutic interest, such as, for instance, paracetamol (IUPAC name: N-(4-hydroxyphenyl)acetamide) and ibuprofen (IUPAC name: (RS)-2-(4-(2-methylpropyl) phenyl) propanoic acid).

### Paracetamol

An object of the invention is therefore a process for preparing paracetamol comprising the following steps of:
a) reacting a compound of formula (II):

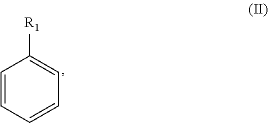

R₁ being a hydroxy group,
   with methane sulfonic acid and a compound of formula (III):

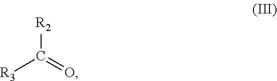

in which R₂ is a methyl group, and R₃ is a hydroxy group;
b) recovering a compound of formula (I)

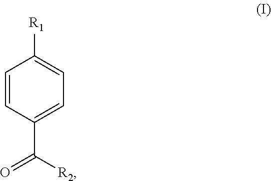

in which R₁ is a hydroxy group, and R₂ is a methyl group;
c) reacting said compound of formula (I) with formic acid and hydrogen peroxide;
d) reacting the mixture obtained after step c) with ammonium acetate and acetic acid; and
e) recovering paracetamol.

Such a process for preparing paracetamol thus comprises the following steps:
a) reacting phenol with methane sulfonic acid and acetic acid;
b) recovering 4-hydroxyacetophenone;
c) reacting 4-hydroxyacetophenone with formic acid and hydrogen peroxide;
d) reacting the mixture obtained after step c) with ammonium acetate and acetic acid; and
e) recovering paracetamol.

In this process, the step c) corresponds to a reaction using “Bayer-Villiger” conditions. In a particular embodiment, 4-hydroxyacetophenone is reacted with formic acid and hydrogen peroxide at room temperature. Preferably 2 to 10 equivalents, more preferably 3 to 7 equivalents, even more preferably 5 equivalents of formic acid relative to 4-hydroxyacetophenone is used. In a further embodiment, 1 to 2 equivalents, preferably 1 to 1.2 equivalents of formic acid relative to 4-hydroxyacetophenone is used.

The step d) corresponds to nucleophilic substitution reaction using ammonium acetate and acetic acid. In a particular embodiment, the mixture obtained after step c) is reacted with ammonium acetate and acetic acid at a temperature between 200 and 250° C., preferably about 230° C.

In a preferred embodiment, the process for preparing paracetamol comprises the following steps of:
a) reacting phenol with methane sulfonic acid and acetic acid at a temperature about 50° C., in which 2.5 equivalents of acetic acid relative to phenol is used;
b) recovering 4-hydroxyacetophenone;
c) reacting 4-hydroxyacetophenone with formic acid and hydrogen peroxide;
d) reacting the mixture obtained after step c) with ammonium acetate and acetic acid; and
e) recovering paracetamol.

The implementation of the step c) “Bayer-Villiger reaction” starting from 4-hydroxyacetophenone using hydrogen peroxide and formic acid can give acetyl hydroquinone as a major product and hydroquinone as a minor product. Hydroquinone can therefore be isolated by any purification methods known from a skilled person. For instance, hydroquinone can be isolated from the mixture acetyl hydroquinone/hydroquinone by hydrolysis and distillation.

In a particular aspect, the process for preparing paracetamol as above defined further comprises a step of purifying the mixture obtained after step c) to recover hydroquinone. Preferably, hydroquinone is purified and isolated with hydrolysis and any distillation methods currently used by a skilled person. Hydroquinone resulting from the purification of the mixture obtained after step c) can therefore be used as an intermediate to provide paracetamol in one chemical step.

A further object of the invention is therefore a process for preparing paracetamol comprising the following steps of:
reacting hydroquinone with ammonium acetate at a temperature ranging from 240 to 300° C. for a period from 1 min to 12 hours in which 1 to 50 equivalents of ammonium acetate relative to hydroquinone are used, wherein the reaction is carried out in the absence of acetic acid; and
recovering paracetamol.

In a preferred embodiment, hydroquinone and ammonium acetate are reacted at a temperature from 240° C. to 300° C. for 10 minutes to 2 hours in which 5 to 30 equivalents of ammonium acetate relative to hydroquinone are used.

In a more preferred embodiment, hydroquinone and ammonium acetate are reacted at a temperature about 280° C. for about 30 minutes in which 20 equivalents of ammonium acetate relative to hydroquinone are used.

In a further more preferred embodiment, hydroquinone and ammonium acetate are reacted at a temperature about 260° C. for about 1 hour in which 10 equivalents of ammonium acetate relative to hydroquinone are used.

A further object of the invention is also a process for preparing paracetamol comprising the following steps of:
reacting hydroquinone with acetamide and water at a temperature ranging from 240 to 300° C. for a period from 1 min to 12 hours in which 1 to 50 equivalents of acetamide and 1 to 50 equivalents of water, relative to hydroquinone are used, wherein the reaction is carried out in the absence of acetic acid; and
recovering paracetamol.

In a preferred embodiment, hydroquinone, acetamide, and water are reacted at a temperature from 240° C. to 300° C. for 10 minutes to 2 hours in which 5 to 30 equivalents of acetamide and 5 to 30 equivalents of water, relative to hydroquinone are used.

In a more preferred embodiment, hydroquinone, acetamide, and water are reacted at a temperature about 260° C. for about 1 hour in which 10 equivalents of acetamide and 10 equivalents of water, relative to hydroquinone are used.

The processes disclosed above for preparing paracetamol starting from hydroquinone using ammonium acetate or acetamide and water allows to provide paracetamol with a high selectivity (>95% even 100%) in a very short time reactional (less than 12 hours, even 1 hour).

Particularly, such processes further comprise a step of recovering ammonium acetate or acetamide for recycling.

In the processes for preparing paracetamol from hydroquinone, the reaction is carried out without acetic acid. The absence of acetic acid allows to improve the conversion rate of paracetamol from hydroquinone while reducing impurities and reactional time. Such processes or methods are therefore well adapted to the industrial scale since they can be implemented with continuous reactor and small industrial material.

### Ibuprofen

A process for preparing ibuprofen comprising the following steps:
a) preparing 1-(4-isobutylphenyl)ethanone as above defined; and
b) obtaining ibuprofen from the intermediate 1-(4-isobutylphenyl)ethanone recovered at step a).

A particular object of the invention is thus a process for preparing ibuprofen comprising the following steps of:
a) reacting a compound of formula (II):

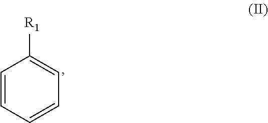

R₁ being an isobutyl group,
   with methane sulfonic acid and a compound of formula (III):

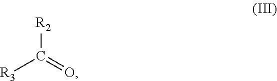

in which R₂ is a methyl group, and R₃ is a —O—CO—CH₃ group;
b) recovering a compound of formula (I)

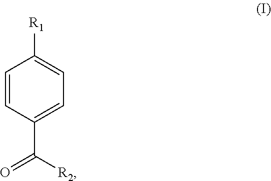

in which R₁ is an isobutyl group, and R₂ is a methyl group; and
c) obtaining ibuprofen from the intermediate 1-(4-isobutylphenyl)ethanone recovered at step b).

Such a process for preparing ibuprofen thus comprises the following steps:
a) reacting 4-isobutylbenzene with methane sulfonic acid and acetic anhydride;
b) recovering 1-(4-isobutylphenyl)ethanone; and
c) obtaining ibuprofen from the intermediate 1-(4-isobutylphenyl)ethanone recovered at step b).

Preferably, such a process comprises the following steps:
a) reacting 4-isobutylbenzene with methane sulfonic acid and acetic anhydride at a temperature about 50° C. in which 2 equivalents of acetic anhydride relative to 4-iso-butylphenyl is used;
b) recovering 1-(4-isobutylphenyl)ethanone; and
c) obtaining ibuprofen from the intermediate 1-(4-isobutylphenyl)ethanone recovered at step b).

The obtention of ibuprofen from the intermediate 1-(4-isobutylphenyl)ethanone is well known from a skilled person and can be performed using several methods, such as those disclosed by James Speight in the Handbook of Industrial Hydrocarbon Processes, page 588-590. Two major chemical ways to obtain ibuprofen from the intermediate 1-(4-isobutylphenyl)ethanone are the Boot process and the Hoechst process. Such pathways comprise the reduction of 1-(4-isobutylacetophenone) to the corresponding alcohol under hydrogen atmosphere with Raney Nickel catalyst followed by a palladium catalyzed carbonylation step as disclosed at scheme 3 of the article from Kjonaas et al. (J. Chem. Educ., 2011, 88, 825-828).

A preferred embodiment of the invention is thus a process for preparing ibuprofen comprising the following steps:
a) preparing 1-(4-isobutylphenyl)ethanone as defined herein;
b) reducing 1-(4-isobutylphenyl)ethanone under hydrogen atmosphere with Raney Nickel to obtain 1-(4-isobutylphenyl)ethanol; and
c) reacting 1-(4-isobutylphenyl)ethanol in a palladium catalyzed carbonylation step; and
d) recovering ibuprofen.

In the same article, Kjonaas et al. further disclose an alternative comprising a four-step synthesis comprising the reduction of 1-(4-isobutylacetophenone) to the corresponding alcohol using sodium borohydride in acetic acid, a nucleophilic substitution to provide the chlorine derivative, a formation of a Grignard reagent followed by carboxylation to provide ibuprofen. A preferred embodiment of the invention is thus a process for preparing ibuprofen comprising the following steps:
a) preparing 1-(4-isobutylphenyl)ethanone as defined herein;
b) reducing 1-(4-isobutylphenyl)ethanone using borohydride in acetic acid to obtain 1-(4-isobutylphenyl)ethanol;
c) reacting 1-(4-isobutylphenyl)ethanol in chlorohydric acid to obtain 1-(4-isobutylphenyl) chloroethane;
e) reacting 1-(4-isobutylphenyl) chloroethane with magnesium to provide the Grignard reagent; and
f) reacting the Grignard reagent with carbon dioxide; and
g) recovering ibuprofen.

Further aspects and advantages of the present invention are disclosed in the following examples, which should be considered as illustrative and not limiting the scope of the present application.

### EXAMPLES

### General Consideration

All reagents and solvents used for synthesis were commercial and supplied by Sigma Aldrich. All the compounds were characterized by spectroscopic data. Nuclear magnetic resonance spectra were recorded on a Brüker DRX 300 or Brüker ALS 300 (1H: 300 MHz, 13C: 75 MHz). Measurements are given in parts per million. Chemical shifts δ are given in ppm. Chemical shifts are given with reference to residual DMSO-d6 central peak: 2.50 ppm for proton, 39.52 ppm for carbon and for residual CDCl₃ central peak: 7.26 ppm for proton, 77.16 ppm for carbon. Abbreviations are defined as follows: s singlet, d doublet, dd doublet of doublets, t triplet, q quadruplet, qt quintet, hex hexuplet, hept heptuplet, m multiplet, br broad. J coupling constants are expressed in Hertz (Hz).

Mass spectra were performed in positive-ion mode on a hybrid quadrupole time-of-flight mass spectrometer (MicroTOFQ-II, Bruker Daltonics, Bremen) with an Electrospray Ionization (ESI) ion source. The flow of spray gas was at 0.6 bar and the capillary voltage was 4.5 kV. The solutions were injected at 180 μL/h in a mixture of solvents (methanol/dichloromethane/water 45/40/15). The mass range of the analysis was 50-1000 m/z, and the calibration was done with sodium formate.

### 1. Acylation of Various Substrates

1.1. Preparation of 4-hydroxyacetophenone

In a round bottom flask were added 10 g of phenol (0.1 mol), 100 mL of methanesulfonic acid (1M) and 15 mL of acetic acid (0.25 mol). After 24 hours at 50° C., 100 ml of water was added at 0° C. and the reaction mixture was extracted with butyl acetate (3×100 mL) then the organic phase was washed with water (3×100 mL) until reached pH 6 (control of the aqueous phase by HPLC), dried Na₂SO₄ and concentrated under vacuum affording the desired product in 83% (average 75-88% yield).

HPLC method: Column C₁₈ (250×4.6 mm, particle size 0.5 μm) •Mobile phase: (H₂O 60+CH₃CN 40)+0.1% v/v H₃PO₄ Isocratic phase •Flow-rate: 1.0 mL·min−1. •Wavelength: 205 nm

¹H NMR of product 300 MHz, CDCl₃: 7.93 (d, 2H), 6.90 (d, 2H), 2.58 (s, 3H).

1.2. Preparation of 1-(4-isobutylphenyl)ethanone

1-(4-isobutylphenyl)ethanone was prepared according to the above protocol at section 1.1. using 4-isobutylbenzene (1 eq.) and acetic anhydride (2 eq.): Yield=80%.

¹H NMR (CDCl₃): d 7.8 (d, 2H, J=7.0 Hz), 7.22 (d, 2H, J=7.0 Hz), 2.50 (s, 3H), 2.45 (d, 2H, J=7.0 Hz), 1.80 (m, 1H), 0.83 (d, 6H, J=7.0 Hz).

¹³C NMR (CDCl₃): 197.8, 147.6, 135, 129.3, 128.3, 45.4, 30.1, 26.5, 22.3.

1.3. Preparation of 1-(4-hydroxy-phenyl)nonan-1-one

1-(4-hydroxy-phenyl) nonan-1-one was prepared according to the above protocol at section 1.1. using phenol and nonanoic acid; Quantitative yield.

¹H (CDCl₃): 7.84 (2H, d, J=8.97 Hz), 6.87 (2H, d, J=8.97 Hz), 2.86 (2H, t, J=7.32 Hz), 1.65 (2H, m), 1.29 (10H, m), 0.79 (3H, t, J=7.14 Hz);

¹³C (CDCl₃): 200.4, 161, 130.8, 129.5, 115.4, 38.4, 31.8, 29.4, 29.1, 24.9, 22.65, 14.09.

1.4. Preparation of 4-hydroxybenzophenone

4-hydroxybenzophenone was prepared according to the above protocol at section 1.1. using phenol and benzoic acid at 60° C.: Yield=60%.

¹H NMR (300 MHz, CDCl₃): δ=7.71-7.65 (m, 4H), 7.56 (tt, J=7.4, 1.7 Hz, 1H), 7.49-7.43 (m, 2H,), 6.88 (tt, J=9.5, 2.4 Hz, 2H).

¹³C NMR (75 MHz, CDCl₃): δ=197.8, 161.0, 138.0, 133.2, 132.2, 129.9, 129.4, 115.5.

1.5. Preparation of 4-methoxybenzophenone

4-methoxy benzophenone was prepared according to the above protocol at section 1.1. using anisole and benzoic acid at 60° C.: Yield=65%.

¹H NMR (CDCl₃): δ=7.83 (d, J=8.8 Hz, 2H), 7.75 (d, J=7.5 Hz, 2H), 7.56 (t, J=7.4 Hz, 1H), 7.46 (t, J=7.5 Hz, 2H), 6.96 (d, J=8.8 Hz, 2H), 3.87 (s, 3H).

¹³C NMR (CDCl₃): δ=195.5, 163.1, 138.2, 132.5, 131.8, 130.0, 129.6, 128.1, 113.5, 55.4.

1.6. Preparation of (4-hydroxyphenyl)-(3,4,5-trihydroxyphenyl) methanone

(4-hydroxyphenyl)-(3,4,5-trihydroxyphenyl) methanone was prepared according to the above protocol at section 1.1. using phenol and 3,4,5-trihydroxybenzoic acid at 120° C.: Yield=55%. 1H NMR (MeOD): δ=7.67 (d, J=7.5Hz, 2H), 6.86 (d, J=7.5 Hz, 2H), 6.82 (s, 2H).

1.7. Preparation of 4-chlorobenzophenone

4-chlorobenzophenone was prepared according to the above protocol at section 1.1. using chlorobenzene and benzoic chloride at 120° C.: Yield=63%.

¹H NMR (300 MHz, CDCl₃): δ 7.78-7.75 (m, 4 H), 7.62-7.59 (m, 1 H), 7.51-7.46 (m, 4 H).

### 2. Preparation of Paracetamol

### 2.1. First Step: Acylation of Phenol

4-hydroxyacetophenone was prepared according to the above protocol at section 1.1.

2.2. Second Step: Baeyer Villiger on 4-hydroxyacetophenone

In a 25 mL three-necked round bottom flask, 10 g (74 mmol) of 4-hydroxyacetophenone and 14 mL (370 mmol: 5 eq.) of formic acid were introduced. 5 mL (50% in water) 1.2 eq. of H₂O₂ was carefully added dropwise by syringe pump during 1 hour at −10° C. After complete addition of hydrogen peroxide, the reaction was keep going back slowly to room temperature over 15 hours. Reaction mixture was extracted with ethyl acetate affording after drying and concentration the desired product was obtained in 86% yield.

¹H NMR of the product, 300 MHz, CDCl₃: 6.94 (d, 2H), 6.78 (d, 2H), 2.29 (s, 3H).

¹³C NMR: 171.3, 153.6, 143.5, 122.2, 116.1, 116.0, 20.9.

### 2.3. Third Step: Nucleophilic Substitution

A mixture of Acetyl hydroquinone/hydroquinone (44.0 g, 0.4 mol, 1 equiv), ammonium acetate (63.0 g, 0.8 mol, 2 equiv) and acetic acid (114 mL, 2 mol, 5 equiv) were added in a 300-mL Parr Instrument reactor equipped with a temperature sensor and a mechanical stirrer. The autoclave was purged with argon and heated to 160° C. (heating mantle) before stirring. The temperature was further increased to 230° C. and the mixture was stirred at this temperature for 15 hours. The reactor was cooled down to room temperature and the homogeneous mixture was transferred to a 250-mL flask (a sample was taken at that stage in order to run HPLC analyses). A distillation set-up was then installed and acetic acid was evaporated under reduced pressure. A total amount of 98 mL was recovered which corresponds to a 85% recovery. The reaction mixture was cooled down to room temperature and the precipitate was filtered, washed twice with water (2×20 mL) and dried to give paracetamol (53.0 g, 88%) as a white solid. HPLC analysis revealed a 99% purity.

¹H NMR (CD₃OD, 500 MHz): δ 7.30 (d, J=8.8 Hz, 2H), 6.72 (d, J=8.8 Hz, 2H), 4.86 (s, 1H), 2.08 (s, 3H): 13C NMR (CD₃OD, 125 MHz): 8 171.3, 155.4, 131.7, 123.3, 116.2, 23.5.

### 2.4. Alternative Strategy Without Acetic Acid

### 2.4.1. Ammonium Acetate

Hydroquinone (5.5 g, 1 equiv) and ammonium acetate (38.5 g, 10 equiv) were added in a 100-mL Parr Instrument reactor equipped with a temperature sensor and a mechanical stirrer. The autoclave was heated to 260° C. The mixture was stirred at this temperature for 1 hour, the observed pressure is 26 bar. At the end of the reaction, the conversion of hydroquinone is up to 90% with a selectivity higher than 95%.

Hydroquinone (2.75 g, 1 equiv) and ammonium acetate (38.5 g, 20 equiv) were added in a 100-mL Parr Instrument reactor equipped with a temperature sensor and a mechanical stirrer. The autoclave was heated to 280° C. The mixture was stirred at this temperature for 30 min, the observed pressure is 32 bar. At the end of the reaction, the conversion of hydroquinone is up to 95%.

¹H NMR (CD₃OD, 500 MHz): δ 7.30 (d, J=8.8 Hz, 2H), 6.72 (d, J=8.8 Hz, 2H), 4.86 (s, 1H), 2.08 (s, 3H): 13C NMR (CD3OD, 125 MHz): 8 171.3, 155.4, 131.7, 123.3, 116.2, 23.5.

### 2.4.2. Mixture Acetamide Water

Hydroquinone (5.5 g, 1 equiv) and acetamide (10 equiv) and water (10 equiv) were added in a 100-mL Parr Instrument reactor equipped with a temperature sensor and a mechanical stirrer. The autoclave was heated to 260° C. The mixture was stirred at this temperature for 1 hour, the observed pressure is 26 bar. At the end of the reaction, the conversion of hydroquinone is up to 90% with a selectivity higher than 95%.

Separation of the reaction mixture give rise to paracetamol as above and 9 equiv of acetamide which could be recycled in another synthesis.

### 2.4.3. EP 2 860 172: Comparative Example (15 H. 220° C.)

The same procedure using Hydroquinone (1 equiv) and ammonium acetate (10 equiv) at 220° C. for 15 hours gives a conversion of hydroquinone of 96% while the selectivity is only 79%.

1. A process for preparing a compound of formula (I): 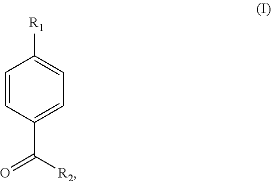
in which:
R₁ is a radical selected in the group consisting of:
a hydroxy group,
a (C₁-C₁₈)alkyl group,
a (C₁-C₆)alkoxy group, and
a halogen group;
R₂ is a radical selected in the group consisting of:
a (C₁-C₁₈)alkyl group,
a phenyl optionally substituted by at least one hydroxy group, and
a (C₁-C₆)alkoxy group;
comprising the following steps of:
a) reacting a compound of formula (II): 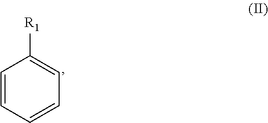
R₁ being such as above defined,
with methane sulfonic acid and a compound of formula (III): 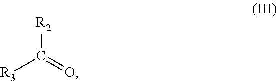
in which:
R₂ is such as above defined, and
R₃ is a radical selected in the group consisting of a hydroxy, a —O—CO—CH₃ group, a (C₁-C₆)alkoxy group, and a chlorine; and
b) recovering said compound of formula (I).

2. The process according to claim 1, wherein R₂ is a radical selected in the group consisting of:
a (C₁-C₁₈)alkyl group,
a phenyl, and
a (C₁-C₆)alkoxy group.

3. The process according to claim 1, wherein the reaction at step a) is carried out at a temperature from 40° C. to 60° C.

4. The process according to claim 1, wherein 1 to 3 equivalents of the compound of formula (III) relative to the compound of formula (II) is used at step a).

5. The process according to claim 1, wherein the compound of formula (I) is such that R₁ is a hydroxy group and R₂ is a methyl group, the compound of formula (II) is such that R₁ is a hydroxy group, and the compound of formula (III) is such that R₂ is a methyl group and R₃ is a hydroxy group.

6. The process according to claim 1, wherein R₁ is hydroxy and the compound of formula (II) is phenol, comprising the following steps of:
a) reacting the phenol with the methane sulfonic acid and acetic acid at a temperature about 50° C. in which 1 to 3 equivalents of the acetic acid relative to the phenol is used; and
b) recovering 4-hydroxyacetophenone.

7. The process according to claim 6, paracetamol, further comprising the following steps of:
c) reacting 4-hydroxyacetophenone with formic acid and hydrogen peroxide;
d) reacting the mixture obtained after step c) with ammonium acetate and a separate batch of acetic acid; and
e) recovering paracetamol.

8. The process according to claim 7, comprising the following steps of:
a) reacting the phenol with the methane sulfonic acid and the acetic acid at a temperature about 50° C., in which 2.5 equivalents of acetic acid relative to the phenol is used;
b) recovering the 4-hydroxyacetophenone;
c) reacting the 4-hydroxyacetophenone with the formic acid and the hydrogen peroxide;
d) reacting the mixture obtained after step c) with the ammonium acetate and the separate batch of acetic acid; and
e) recovering the paracetamol.

9. The process according to claim 7, further comprising a step of purifying the mixture obtained after step c) to recover hydroquinone.

10. A process for preparing paracetamol comprising the following steps of:
reacting hydroquinone with ammonium acetate or acetamide and water at a temperature ranging from 240 to 300° C. for a period from 1 min to 12 hours in which 1 to 50 equivalents of ammonium acetate or acetamide relative to hydroquinone are used, wherein the reaction is carried out in the absence of acetic acid; and
recovering paracetamol.

11. The process for preparing paracetamol according to claim 10, wherein hydroquinone and ammonium acetate are reacted at a temperature about 260° C. for about 1 hour in which 10 equivalents of ammonium acetate relative to hydroquinone are used.

12. The process for preparing paracetamol according to claim 10, wherein hydroquinone, acetamide, and water are reacted at a temperature about 260° C. for about 1 hour in which 10 equivalents of acetamide and 10 equivalents of water, relative to hydroquinone are used.

13. The process according to claim 1, wherein R₁ is a (C₁-C₆)alkyl group, R₂ is a methyl group, and R₃ is a —O—CO—CH₃ group.

14. The process according to claim 1, comprising the following steps:
a) reacting 4-isobutylbenzene with methane sulfonic acid and acetic anhydride at a temperature about 50° C. in which 2 equivalents of acetic anhydride relative to 4-iso-butylphenyl is used; and
b) recovering 1-(4-isobutylphenyl)ethanone.

15. A process for preparing ibuprofen comprising the following steps:
a) preparing 1-(4-isobutylphenyl)ethanone according to claim 14; and
b) obtaining ibuprofen from the intermediate 1-(4-isobutylphenyl)ethanone recovered at step a).

16. The process according to claim 1, wherein:
the compound of formula (I) is such that R₁ is a radical selected in the group consisting of a hydroxy group, a methoxy group, and a chlorine, and R₂ is a radical selected in the group consisting of an octyl group, and a phenyl optionally substituted by at least one hydroxy group,
the compound of formula (II) is such that R₁ is a radical selected in the group consisting of a hydroxy group, a methoxy group, and a chlorine, and
the compound of formula (III) is such that R₂ is a radical selected in the group consisting of an octyl group, and a phenyl optionally substituted by at least one hydroxy group, and R₃ is a hydroxy or a chlorine.

17. The process according to claim 1, wherein the reaction at step a) is carried out at a temperature of about 50° C.

18. The process according to claim 1, wherein 1.5 or 2.5 equivalents of the compound of formula (III) relative to the compound of formula (II) is used at step a).

19. The process according to claim 13, wherein R₁ is an isobutyl group.

20. The process according to claim 16, wherein R₂ is phenyl substituted by with three hydroxy groups.
